(19) United States
(12) Patent Application Publication
(10) Pub. No.: US 2023/0172749 A1
(43) Pub. Date: Jun. 8, 2023

(54) SINGLE TRAY SLEEP APNEA ORAL APPLIANCE

(71) Applicant: R.I.P., LLC, Chatsworth, CA (US)

(72) Inventor: Robin Veis, Malibu, CA (US)

(21) Appl. No.: 17/967,839

(22) Filed: Oct. 17, 2022

Related U.S. Application Data
(63) Continuation-in-part of application No. 16/349,592, filed on May 13, 2019, now abandoned.
(60) Provisional application No. 62/421,172, filed on Nov. 11, 2016.

(51) Int. Cl. A61F 5/56

(52) U.S. Cl. CPC A61F 5/566

(57) ABSTRACT

A single tray sleep apnea oral appliance which includes a hard plastic dental tray, a soft material in the tray for receiving a subject's teeth, a rigid transpalatal bar extending between the left and right sides of the dental tray, and a tongue restrainer having at least two spaced-apart wires extending from the posterior side of the transpalatal bar and a hard plastic tongue-contacting portion attached at the distal end of the wires. The tongue-contacting portion has smooth edges, a rounded posterior end, and a width between its right side and left side greater than the space between the wires, providing greater comfort and user safety, while the wires provide flexibility to vertically position the tongue restrainer and mechanical strength to maintain the tongue restrainer in place once positioned so that a subject's tongue can be prevented from blocking the subject's airway.

### CROSS-REFERENCE TO RELATED APPLICATIONS

This application is a continuation-in-part of U.S. patent application Ser. No. 16/349,592, filed on May 13, 2019, which is the U.S. national stage of International Patent Application No. PCT/US2017/061201 filed on Nov. 11, 2017, which claims the benefit of priority under 35 U.S.C. § 119(e) from U.S. Patent Application No. 62/421,172 filed on Nov. 11, 2016. The disclosures of the foregoing applications are incorporated herein by reference in their entirety.

### BACKGROUND

Sleep apnea is a disorder characterized by abnormal pauses in breathing or instances of abnormally low breathing during sleep. Each pause in breathing, called an apnea, can last from a few seconds to minutes (typically lasting 20 to 40 seconds) and may occur 5 to 30 times or more an hour. Sleep apnea results from a partial-to-complete blockage of a subject's airway. Increased air speed through the airway causes an increase in dynamic pressure and a corresponding drop in static pressure. The decreased static pressure can in some instances draw back the lower jaw and tongue and thereby block the airway. This blockage can increase to the point of becoming complete, which at least temporarily interrupts breathing.

Subjects are generally at greater risk for sleep apnea if they are overweight or have conditions such as diabetes, hypertension, or chronic nasal congestion. There are a variety of factors, however, which can lead to sleep apnea. One factor is the presence of a narrow maxilla and/or mandible in a subject. Maxillary constriction may increase nasal resistance and alter the tongue posture, leading to narrowing of the retroglossal airway. Constriction of the maxilla and/or the mandible generally reduces intraoral air volume and tends to force the tongue back into the posterior airway space, causing the tongue to block a subject's airway during sleep. This leads to obstructive sleep apnea.

A number of oral devices have been designed to address the problem of sleep apnea. Some oral appliances treat snoring and obstructive sleep apnea by repositioning the jaw, usually by moving and holding the mandible in a relatively forward position. Another approach is to through use of a transverse or transpalatal bar to hold the tongue down and open air space in the oral cavity, as described for example in. Other methods used to treat snoring include controlled positive air flow pressure systems (CPAP) which require a nose mask and which are quite uncomfortable, or even surgery.

U.S. Pat. No. 4,669,459 of Spiewak takes a different approach, namely to apply pressure to the soft palate of a subject at the back of the maxilla, i.e. behind the upper teeth of a user of the appliance. In this appliance, a wire projects backwardly from a bridge which is tightly secured to a subject's teeth, and the wire is bent upwardly to apply pressure to the soft palate on the roof of the subject's mouth. The Spiewak appliance is directed to preventing the soft palate from vibrating and thereby inhibit snoring caused by the vibrating soft palate. There remains a need however for an improved oral appliance to treat individuals with sleep apnea.

### SUMMARY

The present invention is a single tray sleep apnea oral appliance for treating snoring and/or sleep apnea of a subject which restricts the backward movement of a user's tongue by applying downward pressure to it and thereby ameliorates or prevents snoring or apnea. In a preferred embodiment, the appliance (10) includes a hard plastic dental tray (100) having an anterior portion (112), a posterior portion (114), a right side (116), a left side (118), an upper side (122), a lower side (124), a buccal side (113), a lingual side (111), an inner surface (117), and an outer surface (119). The upper side and lower side of the dental tray are generally planar, and the tray includes a receptacle (130) bounded by the inner surface (117) of the dental tray. The receptacle can be conformed to receive the dentition of a subject using a hard plastic material, but preferably a soft plastic dental tray portion (150) which can be molded to fit maxillary or mandibular dentition of the subject, such as a thermoplastic material, is retained in the receptacle.

The appliance (10) further includes a hard plastic transpalatal bar (200) having a right side (216), a left side (218), an upper side (213), a lower side (215), an anterior side (212), and a posterior side (214). The transpalatal bar is attached to and extends between the left lingual side (134) of the dental tray and the right lingual side (132) of the dental tray, and is preferably arcuate in shape.

In addition, the appliance (10) includes a tongue restrainer (300) having a proximal end (302) and a distal end (304). The tongue restrainer preferably extends downwardly from the proximal end to the distal end of the tongue restrainer. The tongue restrainer includes at least two spaced-apart metal wires (310) in the proximal end (302) of the tongue restrainer. The proximal end (312) of each wire is affixed to the posterior side (214) of the transpalatal bar (200), such as by being embedded in the transpalatal bar, with the wires extending distally away from the posterior side of the transpalatal bar. In a preferred embodiment, the metal wires are made from an alloy of cobalt, chromium, nickel, molybdenum. The two spaced-apart metal wires (310) can each advantageously include a laterally extending middle portion which forms a partial loop.

The tongue restrainer (300) further includes a hard plastic tongue-contacting portion (350) having an anterior end (352), a posterior end (354), a right side (356), a left side (358), an upper surface (353), a lower surface (355), and radiused edges (351). The spaced-apart wires (310) are affixed to the anterior end (352) of the tongue-contacting portion, such as by being embedded in the tongue-contacting portion, and the anterior end (352), posterior end (354), right side (356), and left side (358) of the lower surface (355) of the tongue-contacting portion have radiused edges. The posterior end (354) is preferably rounded. Preferably, the tongue-contacting portion (350) has a width between the right side (356) of the tongue-contacting portion and the left side (358) of the tongue-contacting portion which is greater than the distance between the two spaced-apart wires (310).

The hard plastic tongue-contacting portion has a length extending between the anterior end and the posterior end and a width extending between the right side and the left side, and the length and width are preferably within 50% of each other. The transpalatal bar can be curved downwardly in order to help maintain the tongue in a relatively forward position, or alternatively it can be straight. Preferably, the dental tray fits the upper, maxillary teeth of the subject.

In a preferred embodiment, the tongue restrainer comprises two wires in order to impede twisting of the hard plastic tongue-contacting portion. The wires are preferably made from an alloy of cobalt, chromium, nickel, molybdenum, and iron in order to better resist stress fractures. The wires are preferably bent downwardly with respect to the plane of the dental tray when worn on the upper dentition of a subject.

### DRAWINGS

FIG. 1 is a top plan view of an embodiment of the present appliance.

FIG. 2 is a bottom plan view of the appliance of FIG. 1.

FIG. 3 is a front elevation view of the appliance of FIG. 1.

FIG. 4A is a rear elevation view of the appliance of FIG. 1.

FIG. 4B is a rear elevation view of the appliance of FIG. 1, without the soft plastic material in the receptacle of the tray.

FIG. 5 is a left side elevation view of the appliance of FIG. 1.

FIG. 6 is a top plan view of an embodiment of a curved transpalatal bar and tongue restrainer of the present appliance.

FIG. 7 is a front elevation view of the curved transpalatal bar and tongue restrainer of FIG. 6.

FIG. 8 is a right side perspective view of the curved transpalatal bar and tongue restrainer of FIG. 6.

FIG. 9 is a top plan view of another embodiment of a curved transpalatal bar and tongue restrainer.

### DETAILED DESCRIPTION

### Definitions

As used herein, the following terms and variations thereof have the meanings given below, unless a different meaning is clearly intended by the context in which such term is used.

The terms “about” and “approximately” refer to a quantity or distance within 10% of the referenced quantity or distance, unless the circumstances of such usage would indicate a different meaning.

“Anterior” means in the direction of or toward or adjacent the front portion (opening) of a subject's mouth when the present appliance is in use by a subject.

“Apnea” and “sleep apnea” refer to a temporary cessation of breathing and/or to instances of shallow or infrequent breathing during sleep, generally caused by a blockage of a subject's airway (referred to as obstructive sleep apnea).

“Arcuate” refers to a surface or shape which is curved, i.e. in the manner of a bow.

“Axial plane” refers to an imaginary plane that divides the body into cranial and caudal (upper and lower) portions.

“Bar” refers to a rigid member having a longitudinal extent greater than its width.

“Buccal” means in the direction of or toward a subject's cheek. In relation to a subject's teeth or a portion of the present appliance adjacent a subject's teeth during use of the appliance, this refers to the side facing the cheek.

“Coronal plane” refers to a hypothetical planar surface that extends through the body from the head to the feet, and divides the body into front and rear halves.

“Coronal” refers to a position or direction which is on or toward the distal end of a tooth (i.e., where the biting surface is located). A coronal surface is thus the biting surface of a tooth, which in posterior teeth is generally referred to as an occlusal surface and on anterior teeth is called an incisal surface.

“Downward” and “downwardly” mean in the direction of or toward a lower portion of a subject's body when the present appliance is in use, i.e. positioned in a subject's mouth. “Upward” and “upwardly” mean in the opposite direction, i.e. in the direction of or toward an upper portion of a subject's body.

“Elongated” refers to a configuration or shape having a length which is longer than its width.

“Hard plastic” refers to a polymer material which melts at a temperature above 212° F. (100° C.) such as poly(methyl methacrylate) (also known as acrylic), polycarbonate, acrylonitrile butadiene styrene (ABS) and others. The hard plastics used for the present appliance are non-toxic and compatible for use in a human subject's mouth, and are sufficiently rigid at body temperature (37° C.) to retain their shape under the pressure exerted by a subject's jaws. Hard plastics used in orthodontic applications are known to the art and can be used in the present appliance.

“Horizontal,” with respect to the present appliance, refers to disposition in a plane approximately perpendicular to the sagittal and/or the coronal plane of a subject, i.e. within 15 degrees of such a perpendicular plane. With respect to a component of the present appliance, “horizontal” refers to such disposition when the appliance is in use by a subject.

“Labial” means in the direction of, toward, or adjacent to a subject's lips. In relation to a subject's teeth or a portion of the present appliance adjacent a subject's teeth during use of the appliance, this refers to the side facing the lips.

“Lateral” means away from the sagittal plane of a subject. With respect to a component of the present appliance, “lateral” refers to such disposition when the appliance is in use by a subject.

“Left” means to the left of the center sagittal plane of a subject, from the perspective of the subject, or to the left of the present appliance when it is worn by a subject.

“Lingual” means in the direction of, toward, or adjacent to a subject's tongue. In relation to a subject's teeth or a portion of the present appliance adjacent a subject's teeth during use of the appliance, this refers to the side facing the tongue.

“Loop” is a shape or structure produced by a curve that bends around and crosses itself. A “partial loop” is a shape or structure produced by a curve that bends around but does not cross itself.

“Lower” refers to the relative position of a component in the present appliance which is closer to or toward a lower portion of a subject's body when the component is being used.

“Mandibular” refers to the lower jaw.

“Mandibular dentition” refers to the teeth of the lower jaw.

“Maxillary” refers to the upper jaw.

“Maxillary dentition” refers to the teeth of the upper jaw.

“Mechanically connected” means physically connected, either through a connection based on direct physical contact or via another intermediate mechanical structure.

“Medial” means toward the center sagittal plane of a subject.

“Occlusal plane” refers to an imaginary plane formed by the occlusal surfaces of the teeth when the jaw is closed. Preferably, the occlusal plane is the imaginary surface that touches the incisal edges of the incisors and the tips of the occluding surfaces of the posterior teeth of a subject.

“Occlusal surface” refers to the surface of a tooth that occludes with or contacts an opposing surface of a tooth in the opposing jaw.

“Orthodontic” refers to a feature or component of an appliance, or an appliance itself, which repositions the teeth and/or jaw(s) of a subject.

“Posterior,” “rear,” and “rearward” mean in the direction of or toward or adjacent the rear portion of a subject's mouth, i.e. the back of the mouth (away from the front teeth), or to a component of the present appliance which is located in this way when worn by a subject.

A “radiused edge” refers to a curved or rounded border between one portion of the outer surface of a component, such as the tongue-contacting portion of the present appliance, and another portion of the outer surface of the component. Radiused edges are not sharp, and do not form a line where two plane faces intersect.

“Receptacle” refers to a portion of the present appliance having a space of sufficient size and volume to receive and retain another article in the receptacle, for example the teeth of a subject.

“Right” means to the right of the center sagittal plane of a subject, from the perspective of the subject, or to the right of the present appliance when it is worn by a subject.

“Sagittal plane” refers to an imaginary plane that travels vertically from the top to the bottom of the body of a subject, dividing it into left and right portions.

“Soft plastic” refers to polymer materials, usually thermoplastic materials, which retain their shape at 100° F. and below, and which become soft (deformable) at a temperature of 212° F. or below.

“Subject” refers to a user of the present appliance, usually a human user.

“Thermoplastic” refers to a polymer material which may be softened by heat and hardened by cooling in a reversible physical process.

“Tray” and “dental tray,” as used herein, refer to a generally U-shaped portion of the present appliance comprising an open area for receiving the maxillary or mandibular teeth of a subject, as the case may be. Trays may comprise cavities or depressions shaped and sized to receive a subject's teeth, as may be formed for example by taking an impression of a subject's teeth.

“Upper” refers to the relative position of a component in the present appliance which is closer to or toward an upper portion of a subject's body when being used.

“Vertical,” with respect to the present appliance, refers to disposition in a plane approximately parallel to the sagittal and/or the coronal plane of a subject, i.e. within 15 degrees of such a parallel plane), when the appliance worn by a subject. Preferably, vertical refers to a direction toward or away from a subject's head or feet.

The term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Oral Appliance

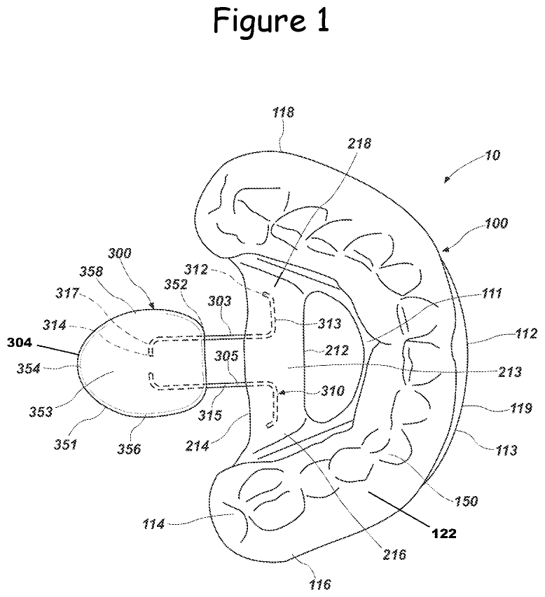

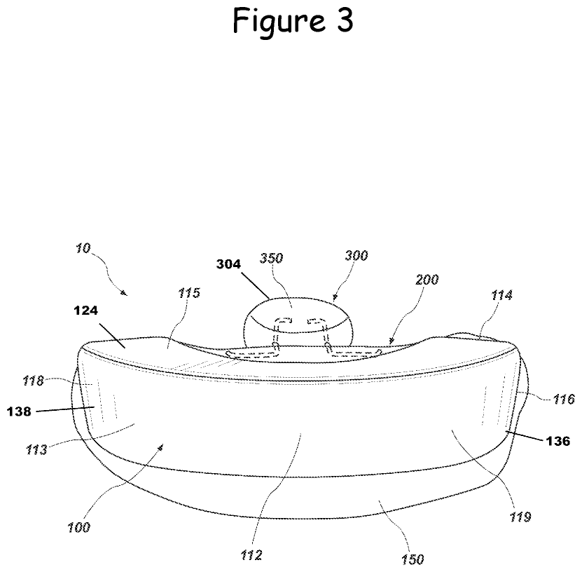

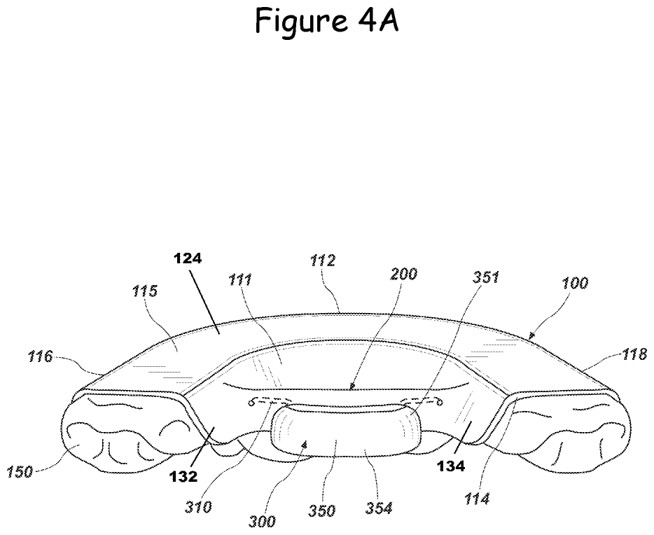

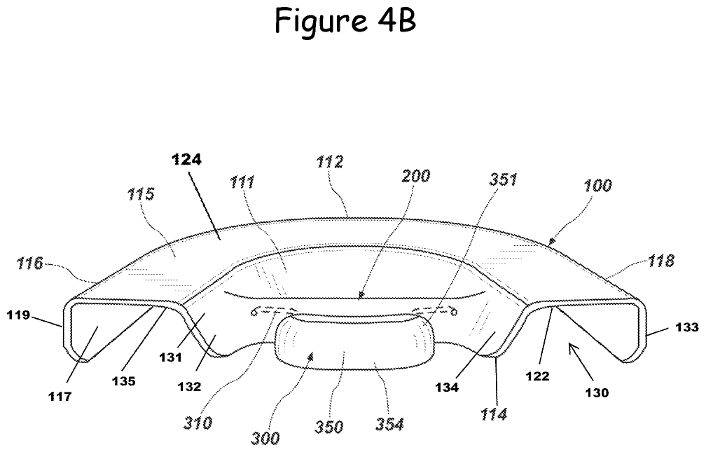

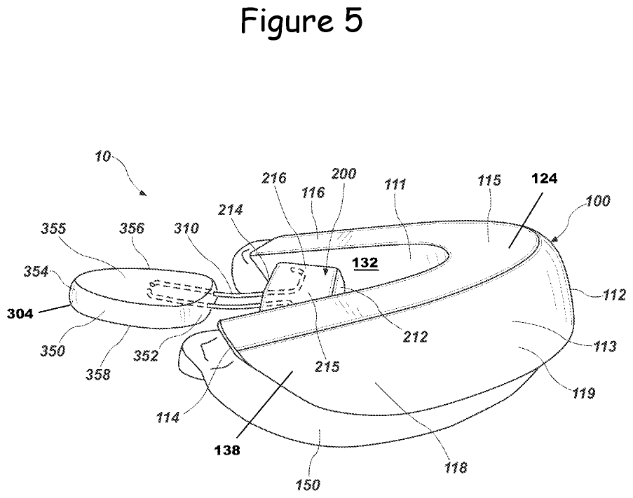

In a preferred embodiment, shown in FIGS. 1-5, the present oral appliance 10 comprises a rigid dental tray 100, a soft plastic dental tray portion 150, a transpalatal bar 200 extending between labial sides of the dental tray 100, and a rearwardly projecting tongue restrainer 300. The dental tray 100 comprises an anterior portion 112, a posterior portion 114, a right side 116, a left side 118, a lingual side 111, a buccal side 113, an inner surface 117, an outer surface 119, an upper side 122, a lower side 124, and a lower surface 115. The tray 100 is generally U-shaped so as to be able to fit over a user's maxillary or mandibular dentition. The tray 100 can be formed from a variety of orally compatible materials, preferably hard plastic polymers. In one embodiment, acrylic is used to form the dental tray 100 of the present appliance.

The dental tray 100 comprises a receptacle 130 bounded by the inner surface 117 of the dental tray 100 for receiving a user's maxillary or mandibular dentition. In the embodiment shown in FIGS. 1-5, the appliance is adapted to receive a user's maxillary dentition. The receptacle in this embodiment comprises a lower surface 135, generally corresponding to the upper side 122 of the dental tray 100, an upwardly extending lingual wall 131 having a right lingual side 132 and a left lingual side 134, and an upwardly extending buccal wall 133 having a right buccal side 136 and a left buccal side 138. In some embodiments, the receptacle 130 can be cast or otherwise formed using a mold of a subject's teeth, so that the receptacle is configured to receive such a user's teeth.

In a preferred embodiment, however, the receptacle 130 comprises a soft plastic dental tray portion 150 which can be thermoplastically shaped to conform to a user's dentition. Such a soft plastic material preferably becomes deformable at a temperature of 212° F. or less, so that the material can be made plastic by being placed in boiling water. Preferably, the soft plastic material of the soft plastic dental tray portion 150 is not deformable at less than 120° F., preferably at not less than 145° F. Thermoplastic polymers, thermosets, thermoplastic elastomers, and other materials known to the art can be used in this embodiment. When thermoplastic materials are used, they must be capable of retaining their shape when used by a subject, and thus preferably remain solid at least at about 100° F., and preferably remain solid at somewhat higher temperatures, such as at 110° F., 120° F., or higher. Such soft plastic material is reversibly retained on the user's maxillary dentition, i.e. so that it can be removed by downward force on the hard plastic dental tray 100.

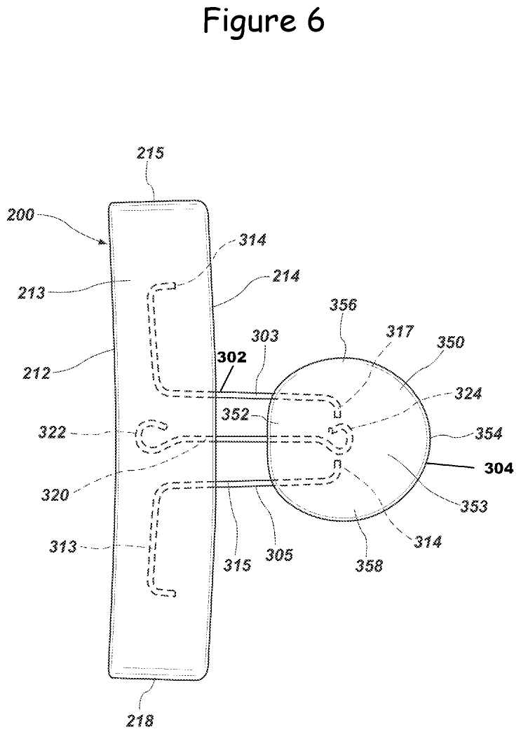

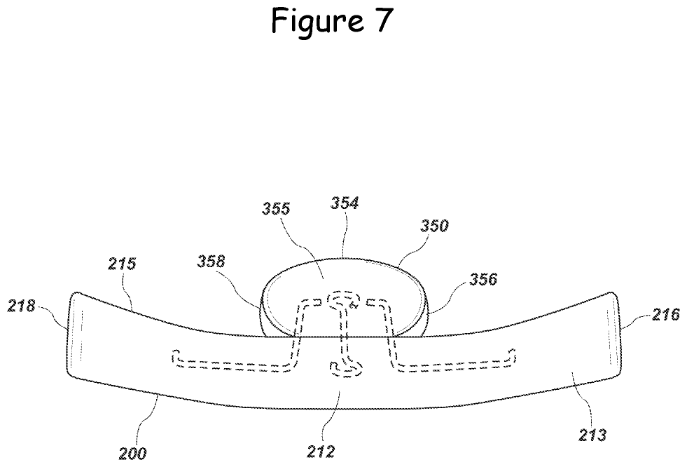

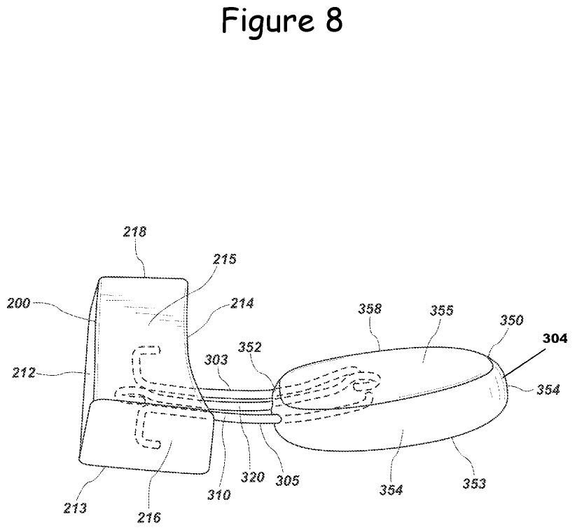

The present appliance 10 also comprises a rigid transpalatal bar 200 which is attached to and extends between the lingual surfaces on the lingual side 111 of the left side 118 and right side 116 of the dental tray 100. Preferably, the transpalatal bar 200 is integrally formed with the dental tray 100, in which case both the tray 100 and transpalatal bar 200 are preferably formed from a hard plastic material. The transpalatal bar 200 comprises an anterior side 212, a posterior side 214, a right side 216, a left side 218, an upper side 213, and a lower side 215. Although the transpalatal bar 200 can be generally horizontal and extend straight between the right lingual side 132 and left lingual side 134 of the lingual wall 131, i.e. with a generally horizontal upper side 213 and lower side 215, in a preferred embodiment, shown in FIGS. 6-9, the transpalatal bar 200 is curved upwardly, toward the palate, in order to provide more free space within the mouth of a user. In this case, the transpalatal bar 200, in particular the upper side 213 and lower side 215 of the transpalatal bar 200, are preferably arcuate, thereby providing space within a user's mouth as well as mechanical strength to the transpalatal bar 200.

The present appliance 10 further includes a tongue restrainer 300 having a proximal end 302 and a distal end 304 comprising one or more wires 310, preferably at least two wires 310, and a tongue-contacting portion 350. The wires 310 preferably include a first wire 303 and a second wire 305, each of which comprise a proximal end 312, a distal end 314, a proximal lateral portion 313, a distal lateral portion 317, and a longitudinal portion 315. The first wire 303 and second wire 305 are preferably spaced apart, i.e. not touching, in order to provide lateral stability to the tongue-contacting portion 350, i.e. resistance to torsion or twisting. The use of 2 wires also provides additional mechanical strength to the tongue restrainer 300. Additional support can be provided by a center wire 320 (shown in FIG. 6) having a proximal end 322 and a distal end 324, which is also preferably spaced apart from the first wire 303 and second wire 305.

The wires 310, 320 of the tongue restrainer 300 extend posteriorly from the transpalatal bar 200, preferably from a center portion of the transpalatal bar 200, and in one embodiment can extend horizontally, i.e. approximately in or parallel to the occlusal plane of a subject when the appliance 10 is in use. However, in preferred embodiments, the wires are angled downwardly, i.e. such that the tongue-contacting portion 350 is positioned lower than the portion of the transpalatal bar 200 from which it extends, in order to better control a user's snoring or apnea.

In a preferred embodiment, the wires are formed from an alloy of cobalt, chromium, nickel, molybdenum, and iron. Such alloys have sufficient tensile strength to support the tongue-contacting portion 350 and resist pressure from a user's tongue, are resistant to oxidation in the aqueous environment of a subject's mouth, and are biocompatible. Importantly, such alloys resist stress fractures caused by the bending of the wire, which may occur during adjustment of the present appliance 10 due to bending and re-bending of the wire, which can introduce stress fractures that can ultimately result in breakage of the wire. Apart from breaking the device 10, breakage of the wire could injure a user if it occurred within the mouth during use. A preferred alloy is a 40Cobalt-20Chromium-16Iron-15Nickel-7Molybdenum alloy wire alloy which meets ASTM standard F1058, such as the wire sold under the trade name ELGILOY (available from Elgiloy Specialty Metals, Sycamore, Ill.). Preferably, 0.036″ (0.9116 mm, 19 gauge) diameter wire is used. Stainless steel, in particular 0.036″ stainless steel wire, or other metal alloys can alternatively be used for the wires.

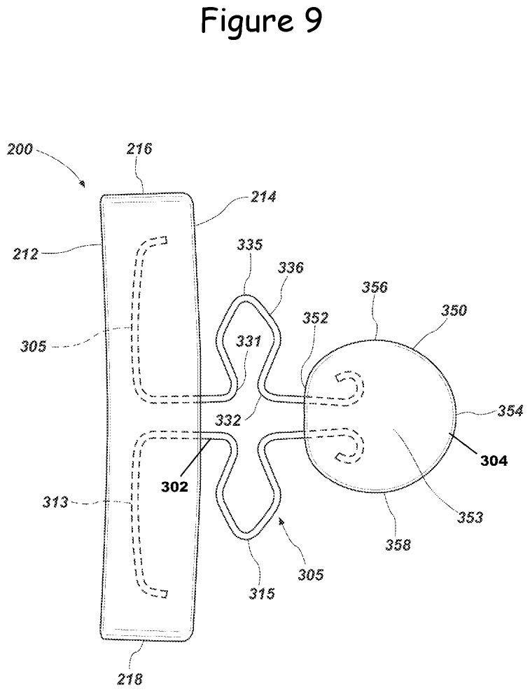

In one embodiment, shown in FIG. 9, as the middle portions of the first wire 303 and second wire 305 extend distally, they also extend laterally outwardly, preferably beyond the right side 356 and left side 358 of the tongue-contacting portion 350, respectively, before curving back inwardly, thereby each forming a partial loop. FIG. 9 illustrates the wires 310 forming two partial loops, right medial loop 336 and left medial loop 338, which have been bent to form laterally extending portions 335. These loops of wire 330 allow the distance between the tongue-contacting portion 350 and the transpalatal bar 200 to be adjusted. The additional lengths of wire which form the partial loops allow the tongue-contacting portion 350 to be moved further from the transpalatal bar 200 by simply pulling the tongue-contacting portion 350 further from the transpalatal bar 200, causing the loops 330 to lengthen (and eventually resulting in the laterally extending portions 335 becoming narrower). Conversely, the tongue-contacting portion 350 can be adjusted to be closer to the transpalatal bar 200 by pushing the tongue-contacting portion 350 closer to the transpalatal bar 200, causing adjacent bends 331 and 332 to come closer together or even overlap.

The tongue-contacting portion 350 is preferably formed from a hard plastic material and comprises an anterior end 352, a posterior end 354, a right side 356, a left side 358, an upper surface 353, and a lower surface 355. In a preferred embodiment, the posterior end 354 is rounded, i.e. has a rounded shape without edges, so that there are no corners or sharp edges between the right side 356 and left side 358 of the tongue-contacting portion 350, in order to provide greater comfort to a user of the present appliance and better avoid injury to the soft tissue of the mouth.

The tongue-contacting portion 350 also preferably comprises radiused edges 351 in order to provide greater user comfort and better avoid the possibility of injury to the soft tissue of the mouth when the appliance 10 is placed in a user's mouth. In particular, the lower surface 355 preferably extends into and/or contacts the anterior end 352, posterior end 354, right side 356, and left side 358 via radiused edges, so that a user's tongue does not contact sharp edges if and when the upper surface of the tongue comes into contact with the tongue-contacting portion 350. Preferably, the upper surface 353 also extends into and/or contacts the anterior end 352, posterior end 354, right side 356, and left side 358 at radiused edges. When both the upper surface 353 and lower surface 355 of the tongue-contacting portion 350 consist of radiused edges, the tongue-contacting portion 350 will presents a smooth surface on all sides.

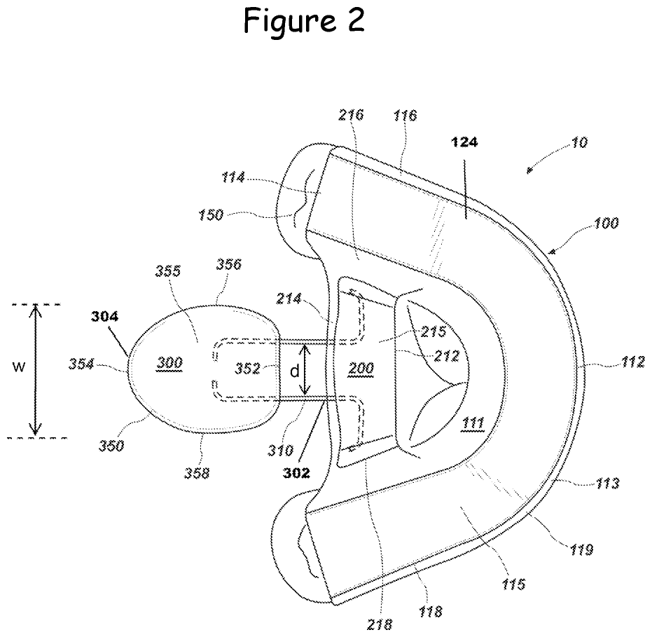

The tongue-contacting portion 350 is further distinguished by having a width “w” between the right side 356 and left side 358 which at its furthest extent is greater (longer) than the distance “d” between the two spaced-apart wires 303, 305 (as shown in FIG. 2). This greater width of the tongue-contacting portion 350 also helps to ensure that a user's tongue will contact the rounded contour of the tongue-contacting portion 350 rather than a hard, thin wire 310 should it fall backward during sleep. The preferred arcuate shape of the transpalatal bar 200 also helps to keep the wires 310 further from a user's tongue.

Preferably the wires 310, 320 are embedded in the transpalatal bar 200 and in the tongue-contacting portion 350, which can be accomplished when these components are made from a plastic material by inserting the wires while the plastic material is heated to a temperature at which it is sufficiently soft to allow the wires to be inserted. Embedding the wires within the transpalatal bar 200 and the tongue-contacting portion 350 prevents the sharp ends of the wire from contacting the soft tissue of a user's mouth, and provides mechanical strength to the present appliance 10 and is therefore preferred, although the components of the present appliance 10 can alternatively be mechanically connected. When the wires 310, 320 are embedded, it is preferred that their proximal and distal ends be curved or otherwise disposed laterally, i.e. at an angle to the direction of the wires when they extend from the transpalatal bar 200 and the tongue-contacting portion 350, in order to better anchor the wires in these components. In the illustrated embodiments, proximal lateral portion 313, distal lateral portion 317, are provided with this configuration. Alternatively, a partial loop can be provided in such wires, as shown in FIG. 6 for example with respect to the proximal end 322 and distal end 324 of the center wire 320.

The features of the tongue-contacting portion 350, i.e. having non-radiused edges, a rounded posterior end 354, and greater width between its right side 356 and left side 358 provide greater comfort and user safety compared to previous oral appliance designs which include a posteriorly extending tongue restrainer. These advantages are paired with the advantageous qualities of using wire in the tongue restrainer 300, which provides both flexibility to vertically position the tongue restrainer 300 with respect to the tray 100 and transpalatal bar 200, as well as providing rigidity once an appropriate vertical positioning is established. The materials and design of the present appliance also lend themselves to use by individuals without specialized knowledge in how to fit oral appliances. In combination, the features of the present appliance 10 provide advantages over appliances known to the art.

### Method of Use

The present appliance 10 is preferably worn over the maxillary dentition by a user during sleep in order to ameliorate or prevent snoring and/or apnea. In alternative embodiments, the appliance 10 can be adapted to be worn over the mandibular dentition.

If apnea or snoring occurs during use, the wires 310 of the tongue restrainer 300 can be bent further downward, in order to place the tongue-contacting portion 350 in a position which is relatively vertically further down, i.e. closer to the resting position of the tongue. Following such repositioning, if the tongue falls backward during sleep, the tongue-contacting portion 350 will contact the user's tongue at a relatively more forward position, in order to prevent the tongue from falling further backward and blocking the user's airway.

In embodiments in which a thermoplastic material is included in the receptacle 130 of the tray 100, in order to fit the appliance 10 to a particular subject's dentition, the soft plastic material of the soft plastic dental tray portion 150 of the receptacle 130 is softened in the manner of a “boil and bite” appliance, namely by placing the appliance 10 in near-boiling water for between several seconds and one minute. The appliance is then placed in the subject's mouth in alignment with the subject's teeth, and the subject is instructed to bite into the softened material to make an impression of the teeth in the softened material. The material is then allowed to cool in the mouth for approximately one minute, after which the appliance is preferably soaked in cold water for an additional minute.

The various features associate with the examples described herein and shown in the accompanying drawings can be implemented in different embodiments and implementations without departing from the scope of the present disclosure. Therefore, although certain specific constructions and arrangements have been described and shown in the accompanying drawings, such embodiments are merely illustrative and not restrictive of the scope of the disclosure, since various other additions and modifications to, and deletions from, the described embodiments will be apparent to one of ordinary skill in the art. Thus, the scope of the disclosure is only determined by the literal language, and legal equivalents, of the claims which follow. All references cited herein are incorporated by reference.

What is claimed is:

1. A single tray sleep apnea oral appliance for treating snoring and/or sleep apnea of a subject by applying downward pressure to the subject's tongue during sleep to maintain the tongue in a forward position, comprising:
a hard plastic dental tray portion having an anterior, a posterior, a right side, a left side, an upper side, a lower side, a buccal side, a lingual side, an inner surface, and an outer surface, wherein the upper side and lower side of the dental tray are planar, the tray further comprising a receptacle bounded by the inner surface of the dental tray;
a soft plastic dental tray portion within the receptacle of the hard plastic dental tray portion for receiving the subject's maxillary dentition;
a hard plastic transpalatal bar having a right side, a left side, an upper side, a lower side, an anterior side, and a posterior side, wherein the transpalatal bar is attached to and extends between the left lingual side of the dental tray and the right lingual side of the dental tray; and
a tongue restrainer having a proximal end and a distal end and comprising:
(a) at least two spaced-apart metal wires in the proximal end of the tongue restrainer, wherein the proximal end of each wire is affixed to the posterior side of the transpalatal bar, the wires extending distally away from the posterior side of the transpalatal bar;
(b) a hard plastic tongue-contacting portion having an anterior end, a posterior end, a right side, a left side, an upper surface, a lower surface, and radiused edges, wherein the spaced-apart wires are affixed to the anterior end of the tongue-contacting portion, and wherein the anterior end, posterior end, right side, and left side of the lower surface have radiused edges,
wherein the proximal ends of the metal wires are embedded in the transpalatal bar, and wherein the distal ends of the metal wires are embedded in the tongue-contacting portion, and
wherein the tongue restrainer extends downwardly from the proximal end of the tongue restrainer to the distal end of the tongue restrainer so as to exert downward pressure to the subject's tongue when the tongue restrainer is contacted by the subject's tongue during sleep.

2. The single tray sleep apnea oral appliance of claim 1, wherein the tongue-contacting portion has a width between the right side of the tongue-contacting portion and the left side of the tongue-contacting portion which is greater than the distance between the two spaced-apart wires.

3. The single tray sleep apnea oral appliance of claim 1, wherein the posterior end of the tongue-contacting portion is rounded.

4. The single tray sleep apnea oral appliance of claim 1, wherein the transpalatal bar is arcuate in shape.

5. The single tray sleep apnea oral appliance of claim 1, wherein the wires are made from an alloy of cobalt, chromium, nickel, molybdenum.

6. The single tray sleep apnea oral appliance of claim 1, wherein the at least two spaced-apart metal wires comprise a first wire and second wire, each wire comprising a laterally extending middle portion which forms a partial loop.

7. The single tray sleep apnea oral appliance of claim 1, wherein the hard plastic dental tray portion is made from acrylic.

8. The single tray sleep apnea oral appliance of claim 1, wherein the soft plastic dental tray portion is formed from a thermoplastic material.

9. A method of treating snoring or sleep apnea, comprising the step of placing the single tray sleep apnea oral appliance of claim 1 on maxillary dentition of a subject in need thereof, thereby ameliorating or preventing snoring or apnea in the subject.
